(19) United States
(12) Patent Application Publication
(10) Pub. No.: US 2006/0099569 A1
(43) Pub. Date: May 11, 2006

(54) Method and device for diagnosing oncological diseases

(76) Inventors: Keiko Akimoto, Kanagawa (JP); Sergei Grigorevich Alekseev, Moscow (RU); Nikolai Borisovich Brandt, Moscow (RU); Galina Aleksandrovna Mironova, Moscow (RU); Hiroshi Akimoto, Kanagawa (JP)

Correspondence Address: DELLETT & WALTERS, P. O. BOX 82788, PORTLAND, OR, 97282-0788, US

(21) Appl. No.: 10/529,586

(22) PCT Filed: Sep. 30, 2003
(86) PCT No.: PCT/RU03/00424

§ 371 Date: Sep. 19, 2005

(30) Foreign Application Priority Data
Sep. 30, 2002 (RU) 2002125918

(51) Int. Cl. C12Q 1/00; G06F 19/00; C12M 1/34

(52) U.S. Cl. 435/4; 435/287.1; 702/19

(57) ABSTRACT

The invention relates to medicine and can be used for diagnosing oncological diseases, manly on early stages thereof and for evaluating a treatment efficiency. The inventive method consists in studying an aqueous solution of native plasma or a native blood serum of a patient with the aid of a laser correlation spectroscopy method (LCS). In order to carry out said method, two solutions are prepared, and alkali is added to one solution, acid being added to another solution. A probability distribution density of fluctuation amplitude of light diffusion intensity in a frequency band of 1-180 Hz is defined for each said solution. A distribution kernel is disclosed, the following characteristic parameters thereof are defined: maximum position, intensity, width and diagnosis index which is equal to a correlation product of said characteristic parameters. When the diagnostic index falls outside the limits of a corresponding interval of allowed values which is admitted as a norm, an oncological disease or the high formation probability thereof are diagnosed. Said invention makes it possible to increase the specificity of the method and the accuracy of measurements and to simplify the measuring unit of the device for diagnosing oncological diseases.

### FIELD OF THE INVENTION

This invention relates to medicine and may be used for diagnosing oncological diseases, especially at an early stage, as well as for assessing the efficiency of treatment conducted.

### PRIOR ART

It is known that the development of pathological processes in a human organism is accompanied by changes in a number of molecular parameters in cells and tissues as well as in the most important of the biological fluids, namely in blood widely used for diagnostic purposes.

Blood is an extremely multicomponent biological fluid. It contains not only low- and high-molecular structures of albumins, globulins, lipoproteins, etc., but also their aggregates and complexes. Furthermore, immunological reactions go on in blood continuously, which are related to the processes of aggregation and disaggregation of immune complexes.

At present, for the purposes of early detection of oncological diseases a rather complex and expensive laboratory equipment, like NMR- and ESR-scanners, is used, which may not be used for mass prophylactic examination (screening) of the population.

The use of immunological markers specific to human tumors (the immune-enzyme analysis of the blood serum) for the purpose of detecting oncological diseases at early stages does not also fulfills the tasks of prophylactic screening of the population due to extremely low diagnostic efficiency at early stages of diseases when the nosology of neoplastic process localization is not fully clear (at stages 1 and 2-5-10%), a rather time-consuming procedure and high cost of tests.

The closest, as to the biophysical essence and the achieved result, to this invention is a method of oncological disease detection, which comprises studying low-concentration aqueous solution of the native plasma or the native blood serum of a patient by a laser correlation spectroscopy method (LCS method) (RU 2132635, A 61 B 5/00, Oct. 7, 1999). The said method is based on experimental evaluation of typical parameters, i.e., maximum frequency (mF), intensity (I) and width (dF) of a selected spectral function kernel of light diffusion intensity, where the dynamics of diffused light fluctuations in low-concentration solutions of the native plasma or the native blood serum under tests is conditioned by the molecular movement of macromolecules of proteins, their aggregates and complexes under the influence of thermal energy K·T, where K is the Boltzmann constant and T is absolute temperature.

At that, the molecular movement in a tested solution represents translation and rotational diffusions, which character and intensity depends, inter alia, on effective dimensions (molecular weight) and the form-factor of light-dissipating molecules as well as their electrostatic interaction conditioned by the value and type of the surface charge spatial distribution.

Theoretic and experimental studies of the said method have shown that an averaged spectral density of fluctuations in the intensity of light diffusion by a tested solution enables to isolate a characteristic spectral kernel, which specifics may be represented by the envelope frequency maximum, the intensity of the gravity center and the half-width of the spectral kernel. At that, in oncological patients values of maximum frequency and those of intensity are lesser and those of half-width are greater than the values of the corresponding parameters typical of non-oncological patients and practically healthy people.

The quantitative and sub-fractional composition of plasma and blood serum as well as the type of intermolecular action, which determine the molecular dynamics in a tested solution, display strong correlation dependence on the homeostasis system, the functional condition of which is directly related to the physiological condition of the main life support biological systems. Therefore, any changes in the physiological condition of an organism, especially any pathological processes, are accompanied by changes in the above physical parameters of the plasma and the blood serum and entail corresponding changes in the molecular dynamics spectral structure in tested solutions.

Since the molecular dynamics spectral structure is adequate to the diffused light spectral structure, it seems possible to decide on the presence or absence of pathological processes in a studied organism, judging by deviations of light-diffusion characteristic parameter values of a tested solution from the values of similar parameters in patients of “practically healthy” type, when the latter are taken as normal. While diagnosing, the complex criterion krG is used, where
krG=F(mF, I, dF).

In the said known inventive method diagnostics is carried out according to the envelope maximum frequency of the spectral kernel and according to a relation between its intensity and its half-width. The clinical tests of the said method, which have been conducted at the clinical radiology department of the Russian Academy of Post-Graduate Education, as headed by A. S. Pavlov, academician of the Russian Academy of Medical Sciences, have shown a rather high efficiency of that express-method. In the scope of the tests conducted the diagnostic efficiency of the express-method has been: according to sensitivity ˜90% and according to specificity (diagnosis efficiency) ˜70% at the average test process time ˜6 minutes.

However, due to the fact that in the said known method a parametric comparison diagnosis one-parameter algorithm is realized, the reliability of diagnosis under the said method is not sufficiently high in all cases.

Furthermore, while fulfilling the screening tasks, the said method has a serious disadvantage conditioned by the fact that diagnosis is based on an experimental evaluation of the absolute values of the diagnostic indices mF, dF and I, which requires frequent calibration of the diagnostic complex instrumental part, which is necessary for ensuring uniform measurements.

The closest, as to the technical essence and the achieved result, to this invention is a device for oncological disease detection, which comprises a laser light source intended for illuminating a dish; a correlation detector composed of: two diffused light receivers and a correlator, the diffused light receivers being installed with the possibility of simultaneously receiving the laser source diffused light beam, as passed through the dish, and transforming light beams into electric signals, the first correlator input being connected to the output of the first receiver and the second correlator input being connected to the output of the second receiver; an analyzer intended for analyzing a correlation signal, the input of the analyzer being connected to the output of the correlator (RU 2132635, A 61 B 5/00, Oct. 7, 1999).

In the above-said technical solution the receivers are made for transforming light signals into analog electric signals, and the correlator is composed of a signal multiplier and a low-pass filter, which are connected in series. As the analyzer a spectral analyzer is used.

The computer laser analyzer of tested blood serum solutions in the known invention is a dual-channel mutually-correlating spectrophotometer of heterodyne oscillator type, which is connected with a microprocessor realizing only one-parameter comparison diagnosis algorithm.

When analyzing a spectrum with the said technical solution, it is necessary to use an optical heterodyne oscillator, which reduces the accuracy of measuring spectral characteristics parameters on the whole and seriously increases the complexity of the optical measuring part of the device.

### DESCRIPTION OF THE INVENTION

The objective of this invention is to develop and create a method and a device for diagnosing oncological diseases, which are to improve the efficiency of diagnostics.

After reaching the said objective it becomes possible to obtain technical results consisting in that the specificity of the method, the reliability of results obtained are improved as well as the hardware of the measuring part of the device for diagnosing oncological diseases is simplified. A technical result that may be obtained after creating a device in accordance with this invention is the creation of a dual-channel laser mutually-coordinating diffused light photometer of heterodyne oscillator type, which ensures conducting multiparametric analysis of a dynamic light-diffusion signal amplitude, enabling to improve measurement accuracy and raise the quality and reliability of diagnostics.

The said technical results are achieved owing to the fact that according to the method of diagnosing oncological diseases, which includes studying a low-concentration aqueous solution of the patient's native plasma or native blood serum by a method of laser correlation spectroscopy (LCS), another low-concentration aqueous solution of the patient's native plasma or native blood serum is prepared; an acid is added to one of the said solutions, and an alkali is added to the other one, a probabilistic distribution density of an amplitude of fluctuations in the light diffusion intensity in the 1-180 Hz frequency band is determined for each solution, the distribution kernel is identified and its characteristic parameters are measured, namely, the maximum position, the intensity, the width and the diagnostic index equal to a correlation product of the said characteristic parameters; and if a value of the said diagnostic index falls out of the corresponding permissible value range taken as normal, an oncological disease or a high probability of an oncological disease is diagnosed.

In order to solve the stated task, in the oncological disease diagnosing device comprising a laser light source intended for illuminating a dish, a correlation detector composed of two diffused light receivers and a correlator, wherein the said diffused light receivers being arranged with the possibility of simultaneously receiving a beam of diffused, passed through the dish, light from the light source and converting light beams into electric signals, the first correlator input being connected to the output of the first receiver and the second correlator input being connected to the output of the second receiver, an analyzer intended for analyzing a correlation signal, the input of the analyzer being connected to the output of the correlator, in accordance with this invention introduced are: a delay unit, two metering units intended for metering an alkali and an acid, respectively, and for their alternate arrangement in the dish, and the analyzer is made so as to ensure a static analysis of a correlation signal amplitude with the possibility of determining a maximum position (mF), an intensity (I), a width (dF) of the distribution density kernel of correlation signal light-diffusion intensity amplitude for an aqueous solution of the native plasma or that of the native blood serum, the said solutions being alternatively arranged in the dish with an alkali and an acid, respectively, and to ensure computation of the diagnostic index krG=mF×dF×I, in the said correlation detector one of the correlator inputs being connected to the output of one of the receivers through the said delay unit, which time of delay being selected so as to be longer than the correlation time of the correlation detector's own hardware noise.

The distinctive feature of this invention in respect of the method is that another low-concentration aqueous solution of the patient's native plasma or native blood serum is prepared, to one of the said solutions an alkali is added, to the other one an acid is added, and this enables to assess the parameters of dynamic light diffusion by tested solutions of plasma or blood serum in the presence of third components, namely, an alkali and an acid, i.e., to take into account the denaturation process of protein macromolecules under their influence, which is of additional prognostic importance. Globular proteins are denatured due to breakages of their disulfide bonds together with the simultaneous increase in the quantity of electro-active SH-groups having a high adsorption power. At that, a probabilistic distribution density of an amplitude of fluctuations in the light diffusion intensity in the 1-180 Hz frequency band is determined for each solution, the distribution kernel is identified and its characteristic parameters are measured, namely, the maximum position, the intensity, the width and the diagnostic index equal to a correlation product of the said characteristic parameters; and if a value of the said diagnostic index falls out of the corresponding permissible value range taken as normal, an oncological disease or a high probability of an oncological disease is diagnosed. In the result, oncological diseases are detected by at least two diagnostic indices, which are correlation products of the characteristic parameters.

Furthermore, the use of the third components, namely, an acid and an alkali, which are added to the low-concentration aqueous solutions, enables to obtain additional diagnostic indices of comparative type (acid/alkali), which results in automatic hardware noise control and eliminates the necessity of frequently calibrating the measuring part of the device in accordance with this invention.

The distinctive feature of this invention in respect of the device consists in using an optical circuit of homodyne type, which, compared to that of heterodyne type, enables to simplify the measuring part of the device greatly, since a circuit fragment, which ensures the process of optical heterodyning, is eliminated.

An additional embodiment of the device is possible, wherein it is advisable that the analyzer should comprise a unit for determining a distribution amplitude density and an intensity of light diffusion, a unit for determining a maximum and a maximum distribution position, a unit for determining a distribution width, a unit for determining the diagnostic criterion, a diagnostic unit, wherein the input of the unit for determining a distribution amplitude density and an intensity of light diffusion being the analyzer input, the first output of the unit for determining a distribution amplitude density and an intensity of light diffusion is connected to the input of the unit for determining a maximum and a maximum distribution position and to the first input of the unit for determining a distribution width, the first input of the unit for determining a maximum and a maximum distribution position being connected to the first input of the unit for determining the diagnostic criterion, the second input of the unit for determining a maximum and a maximum distribution position being connected to the second input of the unit for determining a distribution width, the output of the unit for determining a distribution width being connected to the second input of the unit for determining the diagnostic criterion, the second output of the unit for determining a distribution amplitude density and an intensity of light diffusion being connected to the third input of the unit for determining the diagnostic criterion, the output of the latter being connected to the input of the diagnostic unit.

In addition to the previous embodiment of the device other embodiments are also possible, wherein it is advisable that:
the unit for determining a distribution amplitude density and an intensity of light diffusion should comprise an analog-to-digital converter, a decoder, a right-shift register, a counter of signal discrete components and a group of counters for forming a distribution amplitude density, a summing unit, four AND logical elements, one of them being made of a group of AND logical elements, a multiinput OR logical element, a NOT logical element, wherein the input of the analog-to-digital converter being the input of the unit for determining a distribution amplitude density and an intensity of light diffusion, the digital output of the analog-to-digital converter being connected to the first input of the first AND logical element, and the driving output of the cycle end of the analog-to-digital converter being connected to the second input of the first AND logical element and to the second inputs of the second AND logical elements from the group of AND logical elements, the output of the first AND logical element being connected to the digital inputs of the decoder and the summing unit, each of the decoder outputs being connected, respectively, to the first inputs of the second AND logical elements from the group of AND logical elements, the third inputs of each of the group of the second AND logical elements being connected to the clock circuit, the outputs of the group of AND logical elements being connected, respectively, to the inputs of counters of the group of counters for forming a distribution amplitude density and being connected to the respective inputs of the multiinput OR logical element, which output is connected to the digital input of the counter of signal discrete components, the driving inputs of the counter of signal discrete components, the counter of the group of counters for forming a distribution amplitude density and the summing unit being connected to a zeroing circuit, the digital output of the counter of signal discrete components being connected to the first input of the third AND logical element, the second input of which is connected to the circuit of code for presetting an N sample size, the output of the third AND logical element being the operation terminator circuit for the unit for determining distribution amplitude density and being connected to the first input of the NOT logical element, the digital output of the summing unit being connected to the first input of the fourth AND logical element, the second inputs of the NOT logical element and the fourth AND logical element being connected to the clock circuit, the output of the NOT logical element being connected to the driving input of the analog-to-digital converter, the output of the fourth AND logical element being connected to the input of the right-shift register, the digital outputs of the group of counters for forming a distribution amplitude density being the first output of the unit for determining a distribution amplitude density and a light-diffusion intensity, and the output of the shift register being its second output;
the unit for determining a maximum and a maximum distribution position should comprise: a code multiplexer, three registers, two digital-to-analog converters, a right-shift register, a comparator, a clock pulse counter, four AND logical elements, wherein the digital inputs of the code multiplexer being the input of the unit for determining a maximum and a maximum distribution position, the zeroing circuit being connected to the first driving input of the code multiplexer, to the driving input of the clock pulse counter and to the driving input of the second register, the clock circuit being connected to the first input of the first AND logical element, the operation terminator circuit for the unit for determining distribution amplitude density and light-diffusion intensity being connected, respectively, to the second input of the first AND logical element, the output of the first AND logical element being connected to the second driving input of the code multiplexer and the input of the clock pulse counter, the output of the code multiplexer being connected to the input of the first register, the first output of which is connected to the input of the first digital-to-analog converter and the second output is connected to the first input of the second AND logical element, the output of the first digital-to-analog converter being connected to the first comparison input of the comparator, the output of which is connected to the second input of the second AND logical element and to the first input of the third AND logical element, the output of the second AND logical element being connected to the input of the second register, the first output of which is connected to the input of the second digital-to-analog converter and the second output of the second register is connected to the first input of the fourth AND logical element, the output of the second digital-to-analog converter being connected to the second reference input of the comparator, the second input of the fourth AND logical element being connected to the driving end-of-cycle output of the code multiplexer, the output of the clock pulse counter being connected to the second input of the third AND logical element, the output of which is connected to the input of the third register, the output of the fourth AND logical element being connected to the input of the right-shift register, the output of the third register being the first output of the unit for determining a maximum and a maximum distribution position, the output of the right-shift register being the second output of the unit for determining a maximum and a maximum distribution position, and the driving output of the right-shift register being the operation terminator circuit for the unit for determining a maximum and a maximum distribution position;
the unit for determining a distribution width should comprise: a code multiplexer, three registers, two digital-to-analog converters, a comparator, a clock pulse counter, three AND logical elements, wherein the digital inputs of the code multiplexer being the first input of the unit for determining a distribution width, the zeroing circuit being connected to the first driving input of the code multiplexer, to the driving input of the clock pulse counter and to the driving input of the second register, the clock circuit being connected to the first input of the of the first AND logical element, the operation terminator circuit for the unit for determining a maximum and a maximum distribution position being connected, respectively, to the second input of the first AND logical element, the output of the first AND logical element being connected to the second driving input of the code multiplexer and to the first input of the second AND logical element, the output of the code multiplexer being connected to the input of the first register, the output of which is connected to the input of the first digital-to-analog converter, the output of which is connected to the first comparison input of the comparator, the input of the second register being the second input of the unit for determining of a distribution width, the output of the second register being connected to the input of the second digital-to-analog converter, the output of which is connected to the second reference input of the comparator, the output of the comparator being connected to the second input of the second AND logical element, the output of which is connected to the input of the clock pulse counter, the output of which is connected to the first input of the third AND logical element, the second input of the third AND logical element being connected to the driving end-of-cycle output of the code multiplexer, the output of the third AND logical element being connected to the input of the third register, the output of which is the output of the unit for determining a distribution width, and the end-of-cycle driving output of the code multiplexer being connected to the operation terminator circuit for the unit for determining a distribution width;
the unit for determining the diagnostic criterion should comprise: three AND logical elements, two multipliers, a memory, wherein the first input of the first AND logical element being the third input of the unit for determining the diagnostic criterion, the first input of the second AND logical element being its first input, and the first input of the third AND logical element being its second input, the second input of the first AND logical element being connected to the operation terminator circuit for the unit for determining distribution amplitude density and light-diffusion intensity, the second input of the second AND logical element being connected to the operation terminator circuit for the unit for determining a maximum and a maximum distribution position, the second input if the third AND logical element being connected to the operation terminator circuit for the unit for determining a distribution width, the output of the first AND logical element being connected to the first input of the first multiplier, the output of the second AND logical element being connected to the second input of the first multiplier, the output of which is connected to the first input of the second multiplier, the output of the third AND logical element being connected to the second input of the second multiplier, the output of which is connected to the input of the memory, the output of which is the output of the unit for determining the diagnostic criterion;
the diagnostic unit should comprise one zero-code coincidence logical element and three similar computing devices, each of the latter comprising a digital-to-analog converter, two comparators, a read-only memory with a digital-to-analog converter, the input of which is connected to the output of the read-only memory, an OR logical element, wherein at the input of the third computing device a divider being installed, the output of which is connected to the digital-to-analog converter of that computing device, for two computing devices the inputs of the digital-to-analog converters being the input of the diagnostic unit, and for the third computing device the input of the diagnostic unit comprising two inputs of the divider, the output of which is connected to the digital-to-analog converter of that computing device, for each of the computing devices the output of the digital-to-analog converter being connected to the first comparison input of the first comparator, the first comparison input of the first comparator being connected to the second reference input of the second comparator, the first output for a max value of the read-only memory with the digital-to-analog converter being connected to the second reference input of the first comparator, and the second output for a min value of the read-only memory with the digital-to-analog converter being connected to the first comparison input of the second comparator, the output of the first comparator being connected to the first input of the OR logical element, the output of the second comparator being connected to the second input of the OR logical element, the outputs of the OR logical elements of each of the computing devices being connected, respectively, to the first, the second and the third inputs of the zero-code coincidence logical element.

### DESCRIPTION OF THE DRAWINGS

FIG. 1 represents averaged probabilistic densities of dynamic light-diffusion amplitude distribution by solutions of the examined patients' native plasma or native blood serum, to which an alkali has been added;

FIG. 2 represents averaged probabilistic densities of dynamic light-diffusion amplitude distribution by solutions of the examined patients' native plasma or native blood serum, to which an acid has been added.

FIG. 3 represents a two-parameter diagnostic card of the examined groups of patients, one of which consists of practically healthy volunteers (Group I) and the other consists of oncological patients (Group II), where on the X-axis values of krG1=mF×I×dF in the presence of an alkali are plotted, and on the Y-axis relative values of krG3 of acid/alkali kind are plotted in decibels (dB).

FIG. 4 represents a two-parameter diagnostic card of the examined groups of patients, one of which consists of non-oncological patients (Group III) and the other consists of oncological patients (Group II), where on the X-axis values of krG1=mF×I×dF in the presence of an alkali are plotted, and on the Y-axis relative values of krG3 of acid/alkali kind are plotted in decibels (dB).

FIG. 5 shows the circuit diagram of the device with the block diagram of the statistical analyzer.

FIG. 6 shows the unit for determining a distribution amplitude density and a light-diffusion intensity, as taken from FIG. 5.

FIG. 7 shows the unit for determining a maximum and a maximum position, as taken from FIG. 5.

FIG. 8 shows the unit for determining a distribution width, as taken from FIG. 5.

FIG. 9 shows the unit for determining the diagnostic criterion, as taken from FIG. 5.

FIG. 10 shows the diagnostic unit, as taken from FIG. 5.

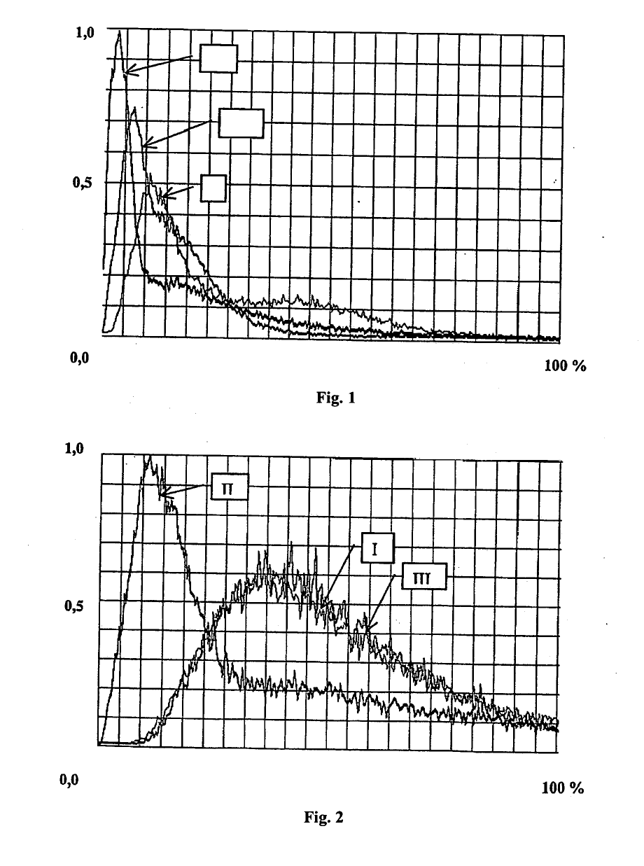

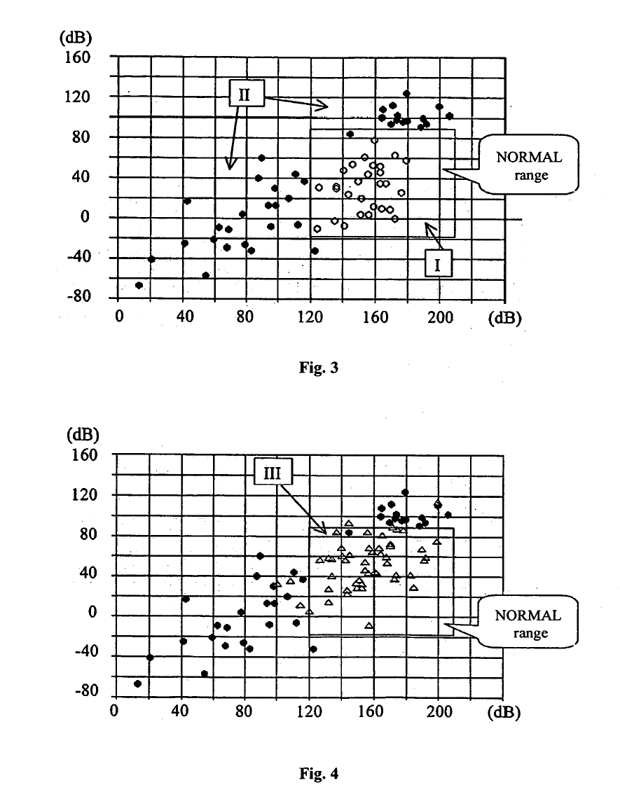

### PREFERRED EMBODIMENT OF THE INVENTION

The procedure of diagnosing oncological diseases comprises the following series of operations:
preparation of two low-concentration aqueous solutions of the patient's native plasma or native blood serum under study by adding 200 microliters of the native plasma or the native blood serum to 5 milliliters of distilled water;
addition to one of the solutions of 100 microliters of a 0.02 M (0.02 moles per 1 liter) aqueous solution of an alkali (NaOH) and the subsequent experimental assessment of the dynamic light-diffusion parameters (mF, dF and I) of that solution;
addition to the other of the said solutions of 100 microliters of a 0.27 M (0.27 moles per 1 liter) aqueous solution of an acid (CH₃COOH) and the subsequent experimental assessment of the dynamic light-diffusion parameters (mF, dF and I) of that solution;
determination of values for the complex diagnostic indices of the type krG=mF×dF×I for low-concentration solutions of the native plasma or the native blood serum, one of the said solutions containing an alkali and the other one—an acid;
comparison of the obtained diagnostic indices krG1, krG2 for solutions of the native plasma or the native blood serum, to which the alkali or the acid, respectively, has been added with the “NORMAL” value range; where the “NORMAL” value range is determined beforehand experimentally, by studying the native plasma or the native blood serum in patients who surely have oncological diseases, or have non-oncological diseases, or are practically healthy;
   determination of the presence or a high probability of an oncological disease, judging by the fact that the obtained values of krG (krG1—for the solution containing the alkali, and krG2—for the solution containing the acid) go beyind the limits of the “NORMAL” allowable range typical of practically healthy people or people having non-oncological diseases.

The clinical tests of the inventive method, which have been carried out in the Moscow Regional Research and Clinical Institute (MONIKI), have shown an increased efficiency of the inventive express-method.

In total, 117 persons have been examined:
Group I comprising 28 persons—volunteers (practically healthy people);
Group II comprising 38 persons—oncological patients (mainly at stages 1-3 of their diseases): breast cancer, thyroid gland cancer, lung cancer, stomach cancer and some other types of oncological diseases;
Group III comprising 51 persons—clinical patients having non-oncological diseases: hypertension, mastopathy, exophthalmic goiter, pneumonia, empyema, emphysema, pneumothorax, gastric ulcer, various benign tumors, etc.

At the alkali action the distribution kernel for the group of non-oncological patients (Group III) is shifted relative to the distribution kernel for the group of practically healthy people (Group I) toward the distribution kernel for the group of oncological patients (Group II).

FIG. 2 represents the averaged (in the scope of each group) probabilistic distribution densities of the amplitudes of dynamic light-diffusion by the solutions of the native plasma or the native blood serum for Groups I-III of the examined patients for the case where the acid has been added to the solutions under study.

When the acid is present, the distribution kernel for the group of non-oncological patients (Group III) practically coincides with the distribution kernel for the group of practically healthy people (Group I), which ensures an increase in the diagnostic efficiency (specificity) of the inventive method.

The specifics of the obtained distributions is naturally reflected by the characteristic parameters, namely, a maximum position (mF), an intensity (I) and a width (dF) of the isolated kernel, and the complex diagnostic criterion (krG) based on them.

The table shows the most typical values of the diagnostic criteria for Groups I-III of the patients examined in the course of the tests. In the scope of the conducted tests, where the limits of the “NORMAL” range are: by the X-axis—[120-210] dB, by the Y-axis—[−10-90], the diagnostic sensitivity and specificity have not been less than 90%.

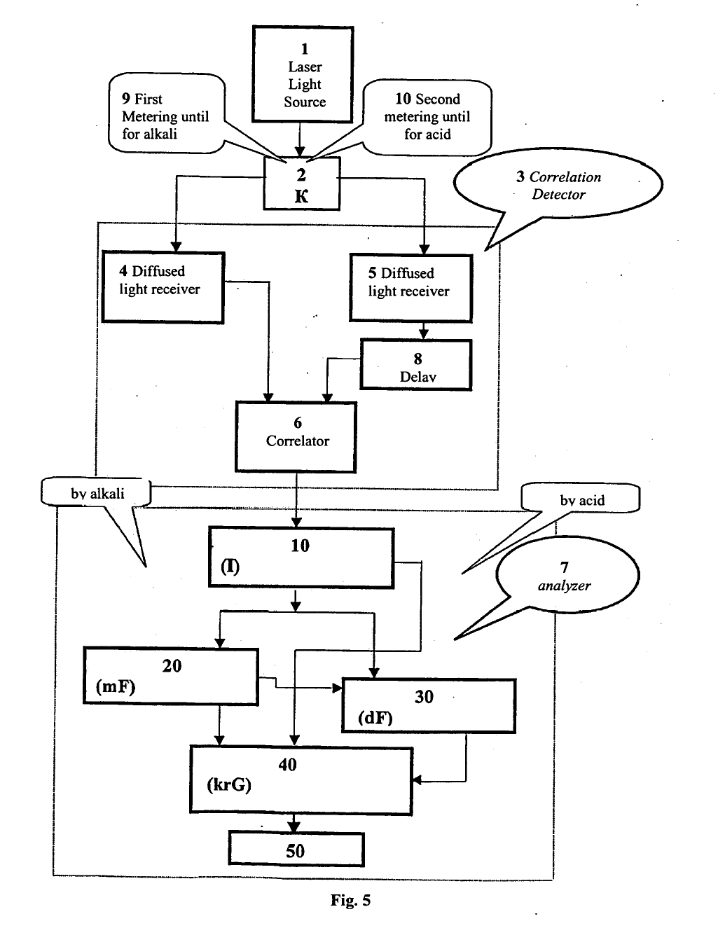

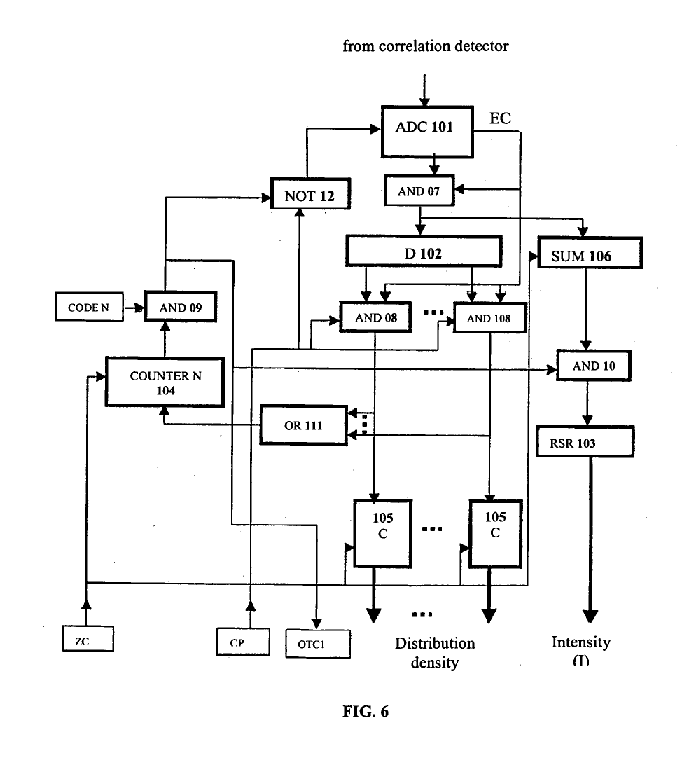

The device for diagnosing oncological diseases (FIG. 5) comprises: a laser light source 1 intended for illuminating the dish 2, a correlation detector 3 composed of two diffused light receivers 4, 5 and a correlator 6. The diffused light receivers 4, 5 (e.g., photo receivers) are arranged with the possibility of simultaneously receiving a beam of diffused, passed through the dish 2, light from the laser source 1 and transforming such light beams into electric signals. The first input of the correlator 6 (composed, e.g., of a signal multiplier and a low-pass filter) is connected to the output of the first receiver 4, and its second input—to the output of the second receiver 5.

The device is completed with a delay unit 8 and two metering units 9, 10. The metering units 9, 10 are intended for metering an alkali and an acid as well as their alternate arrangement in the dish 2. The analyzer 7 is made so as to ensure a statistical analysis of correlation signal amplitudes with the possibility of determining a maximum position (mF), an intensity (I) and a width (dF) of the distribution density kernel of correlation signal light-diffusion intensity amplitude for an aqueous solution of the native plasma or that of the native blood serum, the said solutions being alternatively arranged in the dish with an alkali and an acid, respectively. Furthermore, the analyzer 7 is made with the possibility of ensuring computation of the diagnostic index krG=mF×dF×I for the solution containing an alkali or that containing an acid, respectively. In the correlation detector 3 one input of the correlator 6 is connected to the output of one of the receivers, e.g., to the receiver 5, through the delay unit 8. The delay time is selected so as to be longer than the correlation time of the hardware noise of the correlation detector 3.

The device (FIG. 5) works as follows.

The metering unit 9 is charged with an alkali, and the metering unit 10 is charged with an acid. The metering units 9 and 10 feed these chemicals by, e.g., 100 microliters of a 0.02 M (0.02 moles per 1 liter) aqueous alkali solution (NaOH) and 100 microliters of a 0.27 M (0.27 moles per 1 liter) aqueous acid solution (CH₃COOH). Alternatively, an aqueous solution of the native plasma or an aqueous solution of the native blood serum with the alkali or the acid is placed in the dish 2 and irradiated with the laser source 1. For example, first tests are conducted with the alkali solution for measuring krG1 and then with the acid solution for measuring krG2 in the analyzer 7.

A beam of diffused light is received by the receivers 4, 5, and it is transformed into analog electric signals. One signal is fed to the first input of the correlator 6, and the other signal, which is delayed in the unit 8 by the time t, being longer than the correlation time of the noise of the receivers 4, 5 and the correlator 6, is fed to the second input of the correlator 6.

A correlation signal is fed to the input of the statistical analyzer 7. The statistical analyzer 7 is used for analyzing the amplitude of the correlation signal. The maximum (mF), the intensity (I), the width (dF) of the distribution density kernel of correlation signal light-diffusion intensity amplitude for the correlation signal are determined. Further, krG1 for the alkali is determined, and, after placing the acid solution into the dish 2, krG2 for the acid is determined. Then, the parameters krG1, krG2, krG1/krG2 are compared with the parameters obtained for the alkali solutions and the acid solutions for the normal condition (absence of an oncological disease), by which the presence or absence of a disease, or a possibility of a disease, can be determined.

The device made as it is said above enables to analyze only the correlation signal amplitude, i.e., a heterodyne oscillator is not used. The use of mutual-correlation detection of a diffused light signal having a time delay in this device increases the signal-noise ratio more than 1.4-2 times, which, naturally, improves the statistical reliability when experimentally assessing the characteristic parameters used in the detection of correlation signal amplitudes.

Depending on the equipment used and particular functional layouts used for realizing the analyzer 7, a variety of its block diagrams are possible, and the functional layout of the correlator 7, as presented below, does not exhaust all possibilities of its implementation. It will be understood by those skilled in the art that the functional layouts, as shown below, are just possible, but not the only one embodiment of the analyzer 7. Other variants of the functional layouts of the analyzer 7 may be also used, which are determined by various technical means available for making it.

The analyzer 7 in its additionally claimed embodiment may comprise the unit 10 for determining distribution amplitude density and light-diffusion intensity, the unit 20 for determining a maximum and a maximum distribution position, the unit 30 for determining a distribution width, the unit 40 for determining the diagnostic criterion, and the diagnostic unit 50 (see FIG. 5). The input of the unit 10 is the input of the analyzer 7. The first output of the unit 10 is connected with the input of the unit 20 and to the first input of the unit 30. The first output of the unit 20 is connected to the first input of the unit 40. The second output of the unit 20 is connected to the second input of the unit 30. The output of the unit 30 is connected to the second input of the unit 40. The second output of the unit 10 is connected to the third input of the unit 40. The output of the unit 40 is connected to the input of the unit 50. The units 10, 20, 30, 40, 50 may be also made in accordance with various functional layouts.

The unit 10 for determining distribution amplitude density and light-diffusion intensity (FIG. 6) may comprise an analog-to-digital converter 101 (ADC), a decoder 102 (D), a right-shift register (RSR), a counter 104 of signal discreet components (Counter N), a group of counters 105 (C) for forming distribution amplitude density, a summing unit 106 (SUM), four AND logical elements 107, 108, 109, 110 (AND 107, AND 108, AND 109, AND 110), one of them (AND 108) comprising a group of AND logical elements, a multiinput OR logical element 111 (OR 111), a NOT logical element 112 (NOT 112). The input of ADC 101 is the input of the unit 10. The digital output of ADC 101 is connected with the first input of AND 107 and to the second inputs of the second logical elements AND 108 from the group of AND logical elements. The output of AND 107 is connected to the digital input of the decoder 102 (D) and the summing unit 106 (SUM). Each of the outputs of D 102 is connected, respectively, to the first inputs of AND 108 from the group of AND logical elements. The third inputs of each AND 108 from the group of the second AND logical elements are connected to the clock circuit (CP). The outputs of AND 108 from the group of AND logical elements are connected, respectively, to the inputs of the group of counters 105 (C) for forming distribution amplitude density and are connected, respectively, to the inputs of the multiinput logical element OR 111. The output of OR 111 is connected to the digital input of the counter N 104. The driving inputs of the counter N 104, the group of counters C 105 and the summing unit SUM 106 are connected to the zeroing circuit (ZC) The digital output of the counter N 104 is connected to the first input of AND 109, the second input of which is connected to the circuit of code for presetting an N sample size (code N). The output of AND 109 is the operation terminator circuit (OTC 1) for the unit 10 and is connected to the first input of NOT 112. The digital output of SUM 106 is connected to the first input of AND 110. the second inputs of NOT 112 and AND 110 are connected to the clock circuit (CP).

The output of NOT 112 is connected to the driving input of ADC 101. The output of the fourth logical element AND 110 is connected to the input of the right-shift register 103 (RSR). The digital outputs of the group of C104 for forming distribution amplitude density are the first output of the unit 10 (FIG. 5) for determining distribution amplitude density, and the output of RSR 103 is its second output for light-diffusion intensity.

The unit 10 (FIG. 6) works as follows.

The counter N 104, the group of counters C 106 and the summing unit SUM 106 are zeroed by the initial setting signal (IS). The next clock pulse activates ADC 101. By the EC character of the ADC end-of-cycle, as enabling AND 107 and AND 108, which comes to the second input of AND 107 and the second inputs of AND 108 of the group of AND logical elements, the code of the signal current value comes to SUM 106 and into D 102 through the logical element AND 107. In D 102 it is transformed into a position code gating one logical element AND 108 from the group of AND logical elements and enabling passing the next clock pulse (CP) to the counting inputs of the respective counter C 105 and, through the multiinput logical circuit OR 11, to the input of the counter N 104 as well as to the driving input for activating ADC 101 through the logical element NOT 112. At that, the contents of one corresponding counter C 105 is increased by +1, and summing-up of the current discrete values of the light-diffusion signal is carried out in SUM 106. After that, the unit 10 works similarly until the contents of the counter N 104 is equal to the presetting code N. The presetting code N, for example, comes from the read-only memory (not shown), being a sample size binary code in the power of 2 (e.g., 512, 1024 etc.).

By a coincidence signal, being the enabling signal for the logical element AND 110, which comes from the output of the logical element AND 109, the contents of the summing unit SUM 106 is entered into the shift register RSR 103 where it is right-shifter by a given number of binary digits (a division operation is carried out in RSR 103), the said number being equal to the value of the sample size presetting code N. Simultaneously, a coincidence signal from the output of AND 109, also coming to the logical element NOT 112, locks passage of clock pulses CP to the driving input of ADC 101, which serves as the operation terminator OTC1 for the unit 10, coming to the respective circuit from the output of the logical element AND 109.

In the result of the device operation, at its output the contents of the group of counters C 105 is a sequence of discrete values of the distribution amplitude density components of the light-diffusion signal, and the contents of the right-shift register RSR 103 is the average value of the light diffusion signal intensity I.

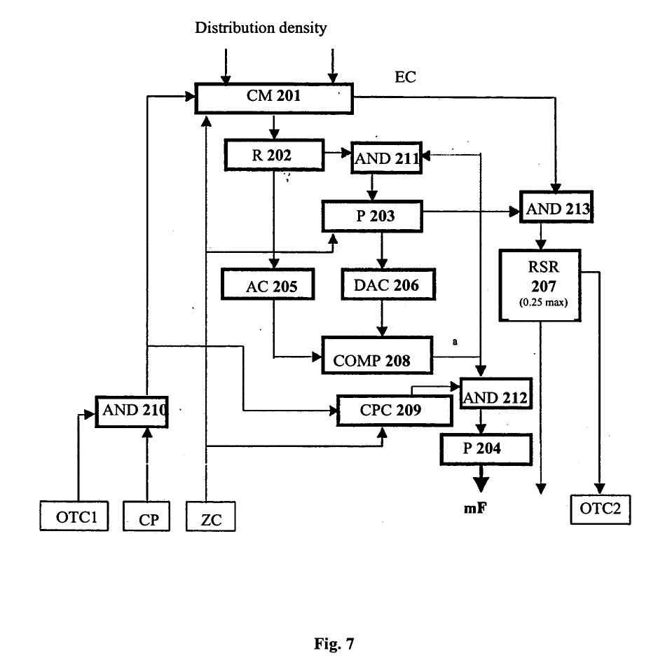

The unit 20 (FIG. 5, FIG. 7) for determining a maximum and a maximum position comprises a code multiplexer 201 (CM), three registers 202, 203, 204 (R), two digital-to-analog converters 205, 205 (DAC), a right-shift register 207 (RSR), a comparator 208 (COMP), a clock pulse counter 209 (CPC), four AND logical elements 210, 211, 212, 213 (AND 210, AND 211, AND 212, AND 213). The digital inputs of CM 201 are the input of the unit 20 (FIG. 5). The zeroing circuit (ZC) (FIG. 7) is connected to the first driving input of CM 201, to the driving input of CPC 209 and to the driving input of the second register R 203. The circuit of the clock (CP) is connected to the first input of the first AND 210. The operation terminator circuit (OTC1) for the unit 10 (FIG. 6) is connected, respectively, to the second input of AND 210 (FIG. 7). The output of AND 210 is connected to the second driving input of CM 201 and to the input of CPC 209. The output of CM 201 is connected to the input of the first R 202. The first output of R 202 is connected to the input of the first DAC 205, and the second output of R 202 is connected to the first input of the second AND 211. The output of the first DAC 205 is connected to the first comparison input of COMP 208. The output of COMP 208 is connected to the second input of the second AND 211 and to the first input of the third AND 212. The output of the second AND 211 is connected to the input of the second register R 203. The first output of R 203 is connected to the input of the second DAC 206, and the second output of R 203 is connected to the first input of the fourth AND 213. The output of the second DAC 206 is connected to the second reference input of the comparator COMP 208. The second input of the fourth AND 213 is connected to the driving end-of cycle (EC) output of the code multiplexer CM 201. The output of CPC 209 is connected to the second input of the third AND 212. The output of AND 212 is connected to the input of the third register R 204. The output of AND 213 is connected to the input of the right-shift register RSR 207. the output of R 204 is the first output of the unit 20 (FIG. 5). The output of RSR 207 is the second output of the unit 20. The driving output of RSR 207 is the operation terminator circuit (OTC2) for the unit 20.

The unit 20 works as follows.

The counter CPC 209, the second register R 203 are zeroed and the distribution density discrete value code multiplexer CM 201 in the volume M is reset. If an enabling signal OTC 1 is present at the input of the first logical element AND 210, the first clock pulse CP increases the contents of the counter CPC 209 by +1, and CM 201 switches the output of the first counter 105 from the group of distribution density counters of the unit 10 (FIG. 6) to the input of the first R 202. After that, codes from the first and the second registers R 202 and R 203 come to the corresponding digital inputs of the first DAC 205 and the second DAC 206, and their analog equivalents come, respectively, to the comparison input (b) and the reference input (a) of the comparator COMP 208. In a case where a signal at the input (b) is higher than a signal at the input (a), a flag appears at the output of the comparator COMP 208, which comes to the enabling inputs of the logical elements AND 211 and AND 212 and ensuring the transfer of the contents of the first register R 202 into the register R 203 and the contents of CPC to the third register R 204. Otherwise, the contents of R 203 and R 204 remains unchanged. When a next clock pulse CP comes, the unit 20 works similarly.

The contents of the registers R 203 and R 204 represents, respectively, values of the distribution density current maximum and the ordinal number of the maximum discrete value determining its position.

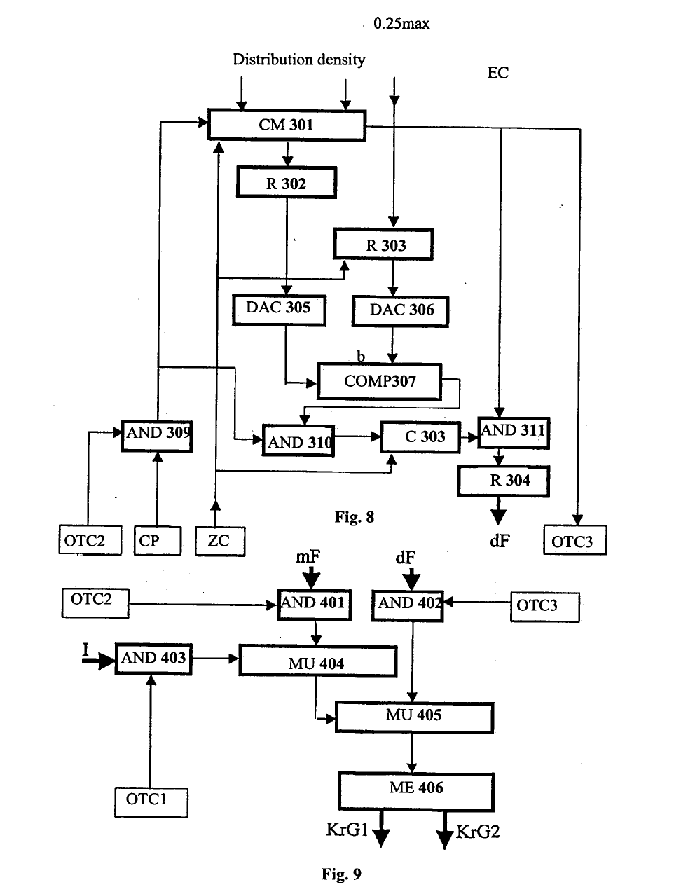

After switching the last distribution density component at the service driving output of CM 201 the switching end-of-cycle (EC) flag is produced, which comes to the second enabling input of the logical element AND 213, ensuring entry of the R 203 contents into the shift register RSR 207 and the subsequent right shift, where each single right shift is equivalent to division of the RSR 207 contents by 2. When being right-shifted by one, two, three binary digits, the contents of RSR 207 represents, respectively, the values of 0.5 max, 0.25 max and 0.125 max of the maximum distribution density value, one of which is used in the unit 30 (FIG. 8) for determining the distribution density width dF at the given level. The signal of shift end in RSR 207, when fed from its driving output, is a operation terminator OTC2 for the unit 20, which activates the unit 30.

The unit 30 for determining a distribution width (FIG. 8) comprises a code multiplexer 301 (CM), three registers 302, 303, 304 (R), two digital-to-analog converters 305, 306 (DAC), a comparator 307 (COMP), a clock pulse counter (CPC) 308, three AND logical elements 309, 310, 311 (AND).

The digital inputs of CM 301 are the first input of the unit 30 (FIG. 5). The zeroing circuit (ZC) is connected to the first driving input of CM 301, to the driving input of C 308 and to the driving input of the second register R 303. The clock circuit (CP) is connected is connected to the first input of the first AND 309. The operation terminator circuit for the unit 20 is connected, respectively, to the second input of the first AND 309. The output of the first AND 309 is connected to the second driving input of CM 301 and to the first input of the second AND 310. The output of the CM 301 is connected to the input of the first R 302. The output of R 302 is connected to the input of the first DAC 305. The output of DAC 305 is connected to the first comparison input of COMP 307. The input of the second R 303 is the second input of the unit 30. The output of the second R 303 is connected to the input of the second DAC 306. The output of DAC 306 is connected to the second reference input of COMP 307. The output of COMP 307 is connected to the second input of the second AND 310. The output of AND 310 is connected to the input of CPC 308. The output of CPC 308 is connected to the first input of the third AND 311. The second input of the third AND 311 is connected to the driving end-of-cycle (EC) output of CM 201. The output of the third AND 311 is connected to the input of the third R 304. The output of R 304 is the output of the unit 30, and the driving end-of-cycle (EC) output of the code multiplexer CM 301 is connected to the operation terminator circuit OTC3 for the unit 30.

The unit 30 works as follows.

The initial setting signal (IS) zeroes the counter CPC 308, R 308, resets CM 301 and enters the value of 0.25 max into the second register R 308. If an enabling signal OTC2 is present at the input of the logical element AND 309, the first clock pulse CP switches by the code multiplexer CM 301 the output of the first counter 105 from the group of distribution density counters of the unit 10 (FIG. 6) to the input of the first register R 302. After that, codes from the registers R 302 and R 303 come to the corresponding digital inputs of the first DAC 305 and the second DAC 306, and their analog equivalents come, respectively, to the comparison input (b) and the reference input (a) of COMP 307. In a case where a signal at the input (b) is higher than a signal at the input (a), a flag appears at the output of the comparator COMP 307, which comes to the enabling second input of the logical element AND 310 ensuring the passage of next CP to the counting input of the counter CPC 308, increasing its content by +1. Otherwise, the contents of CPC 308 remains unchanged. When the next clock pulse CP comes, the unit 30 works similarly. After switching the last distribution density component at the service driving output of CM 201 the switching end-of-cycle (EC) flag is produced, which comes to the second enabling input of the logical element AND 311, ensuring entry of the contents of CPC 308 into the third register R 304.

The contents of the register R 304 represents the number of excesses by the distribution density discreet components of the 0.25 max level, which is the distribution width dF at this set level. The switching end-of-cycle (EC) signal is the operation terminator OTC3 for the unit 30, activating the unit 40 for determining the diagnostic criterion krG.

The unit 40 (FIG. 9) comprises three AND logical elements 401, 402, 403, two multipliers 404, 405 (MU), a memory 406 (ME). The first input of the first AND 401 is the third input of the unit 40, the first input of the second AND 402 is the first input of the unit 40, and the first input of the third AND 403 is the second input of the unit 40. The second input of AND 401 is connected to the operation terminator circuit OTC1 for the unit 10. The second input of AND 402 is connected to the operation terminator circuit OTC2 for the unit 20. The second input of AND 403 is connected to the operation terminator circuit OTC3 for the unit 30. The output of AND 401 is connected to the first input of MU 404. The output of AND 402 is connected to the second input of MU 404. The output of the first MU 404 is connected to the first input of the second MU 405. The output of AND 403 is connected to the second input of MU 405. The output of MU 405 is connected to the input of SD 406. The output of SD 406 is the output of the unit 40.

The unit 40 works as follows.

If the operation terminators OTC1, OTC2, OTC3 for the units 10, 20, 30 are present, values of the characteristic distribution density parameters I, mF and dF come to the respective inputs of the multipliers MU 404 and MU 405 through the respective logical elements AND 401, AND 402, AND 403.

A value of the diagnostic criterion krG is formed at the output of MU 405. The values outputted by MU 405 are stored in SD 406 for alternatively conducted measurements of alkali solutions (krG1) and acid solutions (krG2).

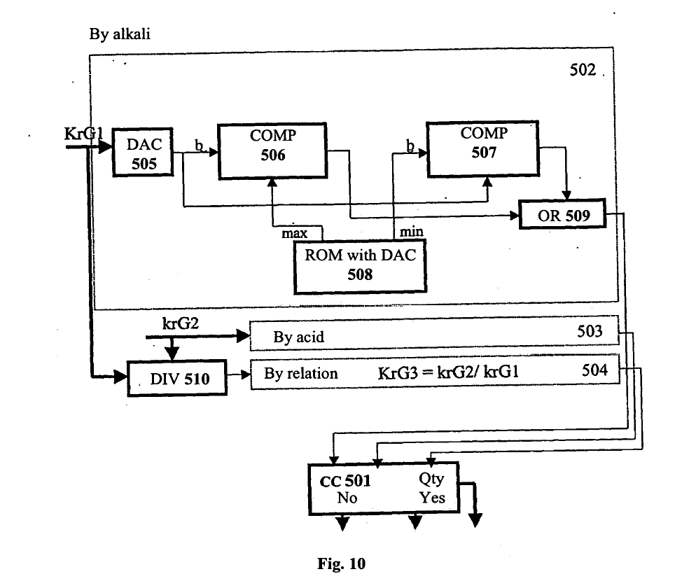

The diagnostic unit 50 (FIG. 10) comprises one logical element of zero code coincidence (CC) and three similar computing devices 502, 503, 504 (CD). Each CD 502, 503, 504 comprises a digital-to-analog converter 505 (DAC), two comparators 506, 507 (COMP), a read-only memory 508 with a digital-to-analog converter (ROM with DAC). The input of that DAC is connected to the output of ROM. Each CD 502, 503, 504 also comprises an OR logical element 509 (OR). At the input of the third computing device 504 a divider 510 (DIV) is arranged. The output of DIV 510 is connected to DAC 505 of the computing device 504 (not shown in FIG. 10 for the sake of simplicity). For the two CD 502, 503 the inputs of their DAC 505 are the input of the unit 50, and for the third CD 504 the input of the diagnostic unit 50 are the two inputs of DIV 510. For each CD 502, 503, 504 the output of DAC 505 is connected to the first comparison input (b) of the first comparator COMP 506. The first comparison input (b) of the first COMP 506 is connected to the second reference input (a) of the second COMP 507. the first output for max values of ROM with DAC is connected to the second reference input (a) of the first COMP 506. The second output for min values of ROM with DAC 508 is connected to the first comparison input (b) of the second COMP 507. The output of the first COMP 506 is connected to the first input of OR 509, the output of the second COMP 507 is connected to the second input of OR 509. The outputs of the OR logical elements of each CD 502, 503, 504 are connected, respectively, to the first, the second and the third inputs of the logical element CC 501.

The diagnostic unit 50 works as follows.

The constant values for the maximum and the minimum NORMAL parameters (max-min) are entered into ROM, forming, naturally, the max output and the min output of ROM with DAC 508. The max output is connected to the second reference input of COMP 506, and the min output is connected to the first comparison input of COMP 507.

A value of the diagnostic criterion krG1, as obtained in the unit 40, comes to the comparison input (b) of the first comparator COMP 506 and the reference input of the second comparator COMP 507. Simultaneously, the NORMAL upper limit (max) value is fed to the reference input (a) of the first comparator COMP 506, and the NORMAL lower limit (min) value is fed to the comparison input (b) of the second comparator COMP 507.

The comparison result signals come to the OR logical element 509, the output of which is connected to the corresponding input of the zero code coincidence logical element CC 501. In a case where a diagnostic criterion value is simultaneously lower than NORMAL max and higher than NORMAL min, at the output of the coincidence circuit CC the flag of comparison to zero appears, being a sign that there is not disease (No). In the other cases the flag of comparison to zero is absent, which, in turn, is a sign that a disease is present (Yes). Testing by alkali is conducted in the first CD 502, testing by acid is conducted in the second CD 503, and the similar testing is conducted in the third CD 504 for the relation krG3=krG2/krG1.

Thus, this diagnostic procedure is conducted according to all diagnostic criteria: krG1 (by alkali), krG2 (by acid) and by their interrelation krG3=krG2/krG1. At that, the number of binary digits different from zero, which corresponds to the number of diagnostic criteria, which values go beyond the NORMAL limits, enables to quantitatively assess the reliability of diagnosing a disease (Qty.), which is an additional positive feature of the inventive solution.

The method and the device for carrying out same in accordance with this invention are characterized by a high degree of automation of the diagnostic process, which completely eliminates the operator's influence on test results, its express character (test time is 8 to 10 minutes), does not require expensive equipment and preparation supply, may be served by one operator of medium qualification.

The method enables to monitor the efficiency of a treatment conducted as well as carry out diagnosis by other biological fluids, e.g., lymph, etc.

The test process completely prevents contacts with an examined patient and is absolutely safe for his/her health.

The method and the device in accordance with this invention may be used in diagnostic centers, clinical and research laboratories, both independently and included on task-oriented diagnostic complexes as a primary means of pre-clinical diagnosis.

TABLE

| | Diagnostic parameter | | Result: | |
|---|---|---|---|---|
| | values | | normal (N), | |
| No. | X (dB) | Y (dB) | risk (R) | DIAGNOSIS |

Group II - oncological patients

| | | | | |
|---|---|---|---|---|
| 1 | 88.9 | 59.7 | R | Breast cancer |
| 2 | 58.6 | −20.9 | R | Breast cancer |
| 3 | 105.9 | 13.3 | R | Breast cancer |
| 4 | 168.8 | 94.5 | R | Thyroid gland cancer |
| 5 | 121.8 | −31.8 | R | Thyroid gland cancer |
| 6 | 94.8 | −8.2 | R | Stomach cancer |
| 7 | 76.9 | 3.9 | R | Stomach cancer |
| 8 | 97.8 | 13.3 | R | Lung cancer |
| 9 | 163.5 | 100.4 | R | Lung cancer |
| 10 | 53.5 | −56.9 | R | Kidney cancer |

Group I - practically healthy (volunteers)

| | | | | |
|---|---|---|---|---|
| 11 | 148.9 | 37.4 | N | Healthy |
| 12 | 162.7 | 51.8 | N | Healthy |
| 13 | 155.1 | 43.5 | N | Healthy |
| 14 | 158.9 | 77.9 | N | Healthy |
| 15 | 143.1 | 24.2 | N | Healthy |
| 16 | 162.3 | 46.2 | N | Healthy |
| 17 | 152.7 | 60.5 | N | Healthy |
| 18 | 145.4 | 54.4 | N | Healthy |
| 19 | 165.8 | 35.5 | N | Healthy |
| 20 | 157.9 | 53.4 | N | Healthy |

Group III - non-oncological patients

| | | | | |
|---|---|---|---|---|
| 21 | 160.1 | 43.8 | N | Mastopathy |
| 22 | 139.6 | 68.1 | N | Thyroid adenoma |
| 23 | 167.2 | 52.8 | N | Exophthalmic goiter |
| 24 | 142.8 | 26.1 | N | Exophthalmic goiter |
| 25 | 155.3 | 84.3 | N | Nodular goiter |
| 26 | 188.8 | 67.1 | N | Stomach ulcer |
| 27 | 140.1 | 59.5 | N | Esophageal diverticulum |
| 28 | 156.3 | 67.5 | N | Pleural empyema |
| 29 | 190.5 | 55.5 | N | Pulmonary emphysema |
| 30 | 148.8 | 28.4 | N | Pulmonary emphysema |

1. A method of diagnosing oncological diseases, including studying a low-concentration aqueous solution of a patient's native plasma or native blood serum by a laser correlation spectroscopy (LCS) method, characterized in that another low-concentration aqueous solution of the patient's native plasma or native blood serum is prepared, an alkali is added to one of the said solutions, and an acid is added to the other one, a probabilistic distribution density of an amplitude of fluctuations in the light diffusion intensity in the 1-180 Hz frequency band is determined for each solution, the distribution kernel is identified and its characteristic parameters are measured, namely, the maximum position, the intensity, the width and the diagnostic index equal to a correlation product of the said characteristic parameters, and if a value of the said diagnostic index falls out of the corresponding permissible value range taken as normal, an oncological disease or a high probability of an oncological disease is diagnosed.

2. The method according to claim 1, characterized in that an additional diagnostic parameter is determined, the relation of the diagnostic parameters obtained when studying the said solutions being used as that additional diagnostic parameter.

3. A device for diagnosing oncological diseases, comprising a laser light source intended for illuminating a dish, a correlation detector composed of two diffused light receivers and a correlator, wherein the said diffused light receivers being arranged with the possibility of simultaneously receiving a beam of diffused, passed through the dish, light from the light source and converting light beams into electric signals, the first correlator input being connected to the output of the first receiver and the second correlator input being connected to the output of the second receiver, an analyzer intended for analyzing a correlation signal, the input of the analyzer being connected to the output of the correlator, characterized in that a delay unit, two metering units intended for metering an alkali and an acid, respectively and for their alternate arrangement in the dish are added, and the analyzer is made so as to ensure a static analysis of correlation signal amplitudes with the possibility of determining a maximum position (mF), an intensity (I), a width (dF) of the distribution density kernel of correlation signal light-diffusion intensity amplitudes for an aqueous solution of the native plasma or that of the native blood serum, the said solutions being alternatively arranged in the dish with an alkali and an acid, respectively, and to ensure computation of the diagnostic index krG=mF×dF×I, in the said correlation detector one of the correlator inputs being connected to the output of one of the receivers through the said delay unit, which time of delay being selected so as to be longer than the correlation time of the correlation detector's own hardware noise.

4. The device according to claim 3, characterized in that the analyzer comprises a unit for determining a distribution amplitude density and an intensity of light diffusion, a unit for determining a maximum and a maximum distribution position, a unit for determining a distribution width, a unit for determining the diagnostic criterion, a diagnostic unit, wherein the input of the unit for determining a distribution amplitude density and an intensity of light diffusion being the analyzer input, the first output of the unit for determining a distribution amplitude density and an intensity of light diffusion is connected to the input of the unit for determining a maximum and a maximum distribution position and to the first input of the unit for determining a distribution width, the first input of the unit for determining a maximum and a maximum distribution position being connected to the first input of the unit for determining the diagnostic criterion, the second input of the unit for determining a maximum and a maximum distribution position being connected to the second input of the unit for determining a distribution width, the output of the unit for determining a distribution width being connected to the second input of the unit for determining the diagnostic criterion, the second output of the unit for determining a distribution amplitude density and an intensity of light diffusion being connected to the third input of the unit for determining the diagnostic criterion, the output of the latter being connected to the input of the diagnostic unit.

5. The device according to claim 4, characterized in that the unit for determining a distribution amplitude density and an intensity of light diffusion comprises an analog-to-digital converter, a decoder, a right-shift register, a counter of signal discrete components and a group of counters for forming a distribution amplitude density, a summing unit, a logical element of coincidence, four AND logical elements, one of them being made of a group of AND logical elements, a multiinput OR logical element, a NOT logical element, wherein the input of the analog-to-digital converter being the input of the unit for determining a distribution amplitude density and an intensity of light diffusion, the digital output of the analog-to-digital converter being connected to the first input of the first AND logical element, and the driving output of the cycle end of the analog-to-digital converter being connected to the second input of the first AND logical element and to the second inputs of the second AND logical elements from the group of AND logical elements, the output of the first AND logical element being connected to the digital inputs of the decoder and the summing unit, each of the decoder outputs being connected, respectively, to the first inputs of the second AND logical elements from the group of AND logical elements, the third inputs of each of the group of the second AND logical elements being connected to the clock circuit, the outputs of the group of AND logical elements being connected, respectively, to the inputs of counters of the group of counters for forming a distribution amplitude density and being connected to the respective inputs of the multiinput OR logical element, which output is connected to the digital input of the counter of signal discrete components, the driving inputs of the counter of signal discrete components, of the group of counters for forming a distribution amplitude density and of the summing unit being connected to a zeroing circuit, the digital output of the counter of signal discrete components being connected to the first input of the third AND logical element, the second input of which is connected to the circuit of code for presetting an N sample size, the output of the third AND logical element being the operation terminator circuit for the unit for determining distribution amplitude density and being connected to the first input of the NOT logical element, the digital output of the summing unit being connected to the first input of the fourth AND logical element, the second inputs of the NOT logical element and the fourth AND logical element being connected to the clock circuit, the output of the NOT logical element being connected to the driving input of the analog-to-digital converter, the output of the fourth AND logical element being connected to the input of the right-shift register, the digital outputs of the group of counters for forming a distribution amplitude density being the first output of the unit for determining a distribution amplitude density and a light-diffusion intensity, and the output of the shift register being its second output.

6. The device according to claim 4, characterized in that the unit for determining a maximum and a maximum distribution position comprises a code multiplexer, three registers, two digital-to-analog converters, a right-shift register, a comparator, a clock pulse counter, four AND logical elements, wherein the digital inputs of the code multiplexer being the input of the unit for determining a maximum and a maximum distribution position, the zeroing circuit being connected to the first driving input of the code multiplexer, to the driving input of the clock pulse counter and to the driving input of the second register, the clock circuit being connected to the first input of the first AND logical element, the operation terminator circuit for the unit for determining distribution amplitude density and light-diffusion intensity being connected, respectively, to the second input of the first AND logical element, the output of the first AND logical element being connected to the second driving input of the code multiplexer and the input of the clock pulse counter, the output of the code multiplexer being connected to the input of the first register, the first output of which is connected to the input of the first digital-to-analog converter and the second output is connected to the first input of the second AND logical element, the output of the first digital-to-analog converter being connected to the first comparison input of the comparator, the output of which is connected to the second input of the second AND logical element and to the first input of the third AND logical element, the output of the second AND logical element being connected to the input of the second register, the first output of which is connected to the input of the second digital-to-analog converter and the second output of the second register is. connected to the first input of the fourth AND logical element, the output of the second digital-to-analog converter being connected to the second reference input of the comparator, the second input of the fourth AND logical element being connected to the driving end-of-cycle output of the code multiplexer, the output of the clock pulse counter being connected to the second input of the third AND logical element, the output of which is connected to the input of the third register, the output of the fourth AND logical element being connected to the input of the right-shift register, the output of the third register being the first output of the unit for determining a maximum and a maximum distribution position, the output of the right-shift register being the second output of the unit for determining a maximum and a maximum distribution position, and the second driving output of the right-shift register being the operation terminator circuit for the unit for determining a maximum and a maximum distribution position.

7. The device according to claim 4, characterized in that the unit for determining a distribution width comprises a code multiplexer, three registers, two digital-to-analog converters, a comparator, a clock pulse counter, three AND logical elements, wherein the digital inputs of the code multiplexer being the first input of the unit for determining a distribution width, the zeroing circuit being connected to the first driving input of the code multiplexer, to the driving input of the clock pulse counter and to the driving input of the second register, the clock circuit being connected to the first input of the of the first AND logical element, the operation terminator circuit for the unit for determining a maximum and a maximum distribution position being connected, respectively, to the second input of the first AND logical element, the output of the first AND logical element being connected to the second driving input of the code multiplexer and to the first input of the second AND logical element, the output of the code multiplexer being connected to the input of the first register, the output of which is connected to the input of the first digital-to-analog converter, the output of which is connected to the first comparison input of the comparator, the input of the second register being the second input of the unit for determining of a distribution width, the output of the second register being connected to the input of the second digital-to-analog converter, the output of which is connected to the second reference input of the comparator, the output of the comparator being connected to the second input of the second AND logical element, the output of which is connected to the input of the clock pulse counter, the output of which is connected to the first input of the third AND logical element, the second input of the third AND logical element being connected to the driving end-of-cycle output of the code multiplexer, the output of the third AND logical element being connected to the input of the third register, the output of which is the output of the unit for determining a distribution width, and the end-of-cycle driving output of the code multiplexer being connected to the operation terminator circuit for the unit for determining a distribution width.

8. The device according to claim 4, characterized in that the unit for determining the diagnostic criterion comprises three AND logical elements, two multipliers, a memory, wherein the first input of the first AND logical element being the third input of the unit for determining the diagnostic criterion, the first input of the second AND logical element being its first input, and the first input of the third AND logical element being its second input, the second input of the first AND logical element being connected to the operation terminator circuit for the unit for determining distribution amplitude density and light-diffusion intensity, the second input of the second AND logical element being connected to the operation terminator circuit for the unit for determining a maximum and a maximum distribution position, the second input if the third AND logical element being connected to the operation terminator circuit for the unit for determining a distribution width, the output of the first AND logical element being connected to the first input of the first multiplier, the output of the second AND logical element being connected to the second input of the first multiplier, the output of which is connected to the first input of the second multiplier, the output of the third AND logical element being connected to the second input of the second multiplier, the output of which is connected to the input of the memory, the output of which is the output of the unit for determining the diagnostic criterion.

9. The device according to claim 4, characterized in that the diagnostic unit comprises one zero-code coincidence logical element and three similar computing devices, each of the latter comprising a digital-to-analog converter, two comparators, a read-only memory with a digital-to-analog converter, the input of which is connected to the output of the read-only memory, an OR logical element, wherein at the input of the third computing device a divider being installed, the output of which is connected to the digital-to-analog converter of that computing device, for two computing devices the inputs of the digital-to-analog converters being the input of the diagnostic unit, and for the third computing device the input of the diagnostic unit comprising two inputs of the divider, the output of which is connected to the digital-to-analog converter of that computing device, for each of the computing devices the output of the digital-to-analog converter being connected to the first comparison input of the first comparator, the first comparison input of the first comparator being connected to the second reference input of the second comparator, the first output for a max value of the read-only memory with the digital-to-analog converter being connected to the second reference input of the first comparator, and the second output for a min value of the read-only memory with the digital-to-analog converter being connected to the first comparison input of the second comparator, the output of the first comparator being connected to the first input of the OR logical element, the output of the second comparator being connected to the second input of the OR logical element, the outputs of the OR logical elements of each of the computing devices being connected, respectively, to the first, the second and the third inputs of the zero-code coincidence logical element.
